(19) United States
(12) Patent Application Publication
(10) Pub. No.: US 2006/0251580 A1
(43) Pub. Date: Nov. 9, 2006

(54) Lectin conjugates

(75) Inventors: Bernhard Keppler, Gaweinstal (AT); Paul Debbage, Telfes (AT); Wolfgang Buchberger, Siegmunds (AT)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: Faustus Forschungs CIE.Translational Cancer Research GmbH

(21) Appl. No.: 11/294,963

(22) Filed: Dec. 6, 2005

Related U.S. Application Data
(63) Continuation of application No. PCT/EP04/06141, filed on Jun. 7, 2004.

(30) Foreign Application Priority Data
Jun. 6, 2003 (DE) 103 25 752.7

(51) Int. Cl. A61K 51/00; A61K 49/10; C07K 14/47; C07K 16/46

(52) U.S. Cl. 424/1.49; 424/9.34; 530/388.8; 530/391.1; 530/396

(57) ABSTRACT

A conjugate includes at least one target-seeking unit, which specifically binds to receptors on the surface of endothelial cells, and at least one effector unit which is coupled to the unit by a linker. The effector unit exhibits at least one signal unit and optionally at least one therapeutic active substance. The target-seeking unit includes a lectin or a fragment or derivative thereof, wherein the lectin is not L-selectin and the signal unit includes a lanthanide ion.

### CROSS-REFERENCE TO RELATED APPLICATIONS

This application is a continuation of International Application No. PCT/EP2004/006141, filed Jun. 7, 2004, which was published in the German language on Dec. 16, 2004, under International Publication No. WO 2004/108747 A2 and the disclosure of which is incorporated herein by reference.

### BACKGROUND OF THE INVENTION

The invention relates to a conjugate comprising at least one target-seeking unit, which bonds specifically to receptors on the surface of endothelial cells, and at least one effector unit coupled to the unit by a linker. The invention also relates to compositions which contain the conjugates, as well as to their use and the manufacture of the conjugates.

All cells possess special surface molecules, which, for example, facilitate cell-cell interactions and similar processes. This applies accordingly also to the cells of the vascular endothelium. A large proportion of these surface molecules consist of proteoglycans and glycoproteins, the protein structure of which is present more or less strongly glycosylated. The respective expression of oligosaccharides varies from species to species, but also from organ to organ. Furthermore, this so-called glycocalyx changes in a characteristic manner also within the scope of development or modification of the endothelium due to pathophysiological processes. Consequently, even the functional states of the tissue situated below it are signalled through the surface of the endothelial cells.

The (patho)physiological processes, which lead to a modification of the glycocalyx, include, for example, inflammation reactions, the effect of hormones, the reaction to invading organisms, such as for example viruses and in particular modifications in the glycocalyx due to proliferating cells, for example as they occur during angiogenesis. The processes which lead to a proliferation of the endothelium include the new formation of tissue within the scope of wound healing as well as the uncontrolled proliferation of cells during tumor growth and during the metastatic propagation of tumor cells.

These markings on the cell surface of the vascular endothelium, which deviate from the normal state, can therefore be detected, i.e. used for the diagnosis of pathophysiological states of the tissue located beneath, but furthermore they can also be used for targeted transport of therapeutic active substances when they are bound to a suitable carrier molecule. In particular the large variability of possible structures, which arises with the coupling of a given number of sugar molecules and which exceeds the variability of structures formed from amino acids by some magnitudes, enables the representation of the various stages in the development of a cell and also of the respective active functions.

Repeated attempts have been made to use certain markers on the vascular endothelium, which show pathological changes, for the targeted transport of active substances or for the transport of particles, which can be used for diagnostic purposes. Monoclonal antibodies, for example, are often used as target-seeking particles. The general use of this technology is made more difficult due to the low specificity of the structures used or also due to causing an immune response. It is precisely within the scope of diagnostic methods that the immunogenic effect and the destruction of the marked cells due to the body's immune response represent a serious problem with the use of antibodies. Also target structures on the cells of the seat of the disease itself have often been described as the target for a target-seeking particle, whereby here in particular the difficult access to the diseased location should be mentioned as a disadvantage with systematic application.

The natural ligands for the glycoproteins and proteoglycans of the glycocalyx are the lectins. Lectins are proteins or glycoproteins which possess a strong affinity for the sugar structures of the glycocalyx. The structures of the lectins vary in a wide range, but the common feature is that proteins are always involved. A further common feature of lectins is that they bind preferably to carbohydrates with high affinity specifically and reversibly. Here, the affinity of the binding to oligosaccharides is many times higher than the affinity of the binding to monosaccharides. Consequently, the different spatial and also functionally temporal formation of oligosaccharides and of the endothelial cell surface facilitate spatial specific linking of the lectins. Apart from this very favourable property with regard to the selectivity, also the availability of these protein structures from various sources is very favourable.

International application publication No. WO 01/17566 reveals a contrast medium for showing changes in lymph nodes, inflammation processes or pathological changes, which are linked to the specific expression of endothelial and/or leucocytic ligands. With this contrast medium a receptor for specifically exprimed endothelial ligands, specifically L-selectin or an L-selectin derivative, is coupled to a signal unit in a defined alignment.

International application publication No. WO 85/01442 describes various conjugates of lectins, selected from peanut lectin, lectin extract from orange skins, Maclura pomifera lectin, Dolichos biflorus agglutinin and soya bean agglutinin with either a therapeutic agent or a radioactive marking, which is to be used for cancer therapy or for the detection of tumor cells.

### BRIEF SUMMARY OF THE INVENTION

In contrast, the object of the invention is the provision of new types of lectin conjugates, which facilitate the diagnosis of pathophysiological changes of the glycocalyx in a particularly effective manner having a low toxic load on the body and which furthermore can optionally also be simply and effectively coupled to therapeutic active substances. The latter type of embodiment facilitates not only a targeted transport of active substance, but also tracing of the active substance accumulation in the patient's body.

This object is solved according to the invention by conjugates which comprise at least one target-seeking unit, which bonds specifically to receptors on the surface of endothelial cells, and at least one effector unit coupled to the unit by a linker, which comprises at least one signal unit as well as optionally at least one therapeutic active substance and are thereby characterized in that the target-seeking unit comprises a lectin or a fragment or derivative thereof, wherein the lectin is not L-selectin and the signal unit comprises a lanthanide ion.

Fragments of lectins in the sense of the invention represent parts of naturally occurring lectins, which preferably possess the binding specificity of the naturally occurring form.

Derivatives of lectins in the sense of the invention represent preferably chemically modified lectins, which possess the binding specificity of the unmodified lectins. An example for the chemical modification of lectins is biotinylation.

Chemical modifications of lectins can comprise according to the invention:
(i) radioactive modifications, e.g. radioactive phosphorylation or radioactive marking with sulphur, hydrogen, carbon, nitrogen,
(ii) colored groups (e.g. digoxygenin, etc.),
(iii) fluorescent groups (e.g. fluorescein, etc.),
(iv) chemoluminescent groups, and/or
(v) groups for immobilization on fixed phases (e.g. biotin, streptavidin, streptag),
(vi) marking with materials having a high electron density, e.g. gold,
   or combinations of modifications according to two or more of the modifications quoted under (i) to (vi). These modifications can be verified with the aid of reactions known to a person skilled in the art.

In a further aspect of the invention conjugates are provided, comprising at least one target-seeking unit, which bonds specifically to receptors on the surface of endothelial cells, and at least one effector unit coupled by a linker to the unit, which comprises at least one therapeutic active substance and are characterized in that the target seeking unit comprises a lectin or a fragment or derivative thereof, wherein the lectin is not peanut lectin, lectin extract of orange peel, Maclura pomifera lectin, Dolichos biflorus agglutinin or soya bean agglutinin.

The invention also comprises methods for producing these conjugates as well as the use of the conjugates in diagnostic and/or therapeutic methods.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a concentration/effect curve of Compound 1 (LEA-DTPA-Gd) in comparison to omniscan [aqua[5,8-bis(carboxymethyl)-11-[₂,-(methylamino)-2-oxo-ethyl]-3-oxo-2,5,8,11-tetraazatridecane-13-oato-(3-)-N₅,N₈,N₁₁,O₃,O₅,O₈,O₁₁,O₁₃]-gadoliniumhydrate and magnevist [1-desoxy-1-(methylamino)-D-glucitol-dihydrogen-N,N-bis[2-[bis(carboxymethyl)amino]ethyl]-glycinato-(5-)-]gadolinate(2--)(2:1)]; the relative change of signal is referred to the signal value of PBS.

FIG. 2 is a series of MRI images using the latex LEA conjugate 3.4 as contrast medium and the spin echo technique in the human Vena saphena magna: Vein (control), without contrast medium, vein can be recognised in negative contrast; vein (1 week), with contrast medium, vein can be seen in the plan view at the right edge of the small tube; vein (fresh), with contrast medium, plan view on the vein cross-section; PBS as negative control; magnevist as positive control.

FIGS. 3A-3D are MRI images of hepatic vessels of the mouse using the latex LEA conjugate 3.4 as contrast medium, where:

FIG. 3A shows hepatic vessels of the mouse in negative contrast, no contrast medium.

FIG. 3B demonstrates substance 3.4 as contrast medium, hepatic vessels of the mouse are white, and the contrast medium is situated in the vessels (concentration 5.75 mg/ml).

FIG. 3C shows contrast medium in the mouse rinsed out with 1 ml of PBS, vessels are still slightly white in color.

FIG. 3D shows contrast medium rinsed out with 4 ml of PBS, hepatic vessels are still white and contain contrast medium.

### DETAILED DESCRIPTION OF THE INVENTION

Through the use of a conjugate according to the invention, formed from a target-seeking unit and an effector unit matched to the respective task, pathophysiological changes can be rendered detectable and verifiable through the exploitation of the specific interaction between the glycocalyx modified by these pathophysiological changes and the lectins used as the target-seeking unit and optionally an active substance can be transported to the location of the disease and the accumulation of the active substance in the patient's body can be traced.

Apart from the specific carbohydrate structures or specific lectins, which are exprimed from the pathologically changed endothelial cells, the characteristic glycocalyx can also be marked by particles deposited on the vessel wall, such as for example, plaque, arteriosclerosis or a biofilm. Also the fact that the actual surface of the blood vessel is covered by deposits on it and the glycocalyx can therefore no longer be registered can be taken as an indication of a change in the blood vessel and can point to pathophysiological changes. Furthermore, with the conjugates according to the invention also characteristic glycans of the tissue located beneath the blood vessel can be formed under some circumstances if it has been exposed due to pathological processes with partial loss of the vascular tissue.

Preferably, the carbohydrate structures of the glycocalyx of the vascular vessel wall acting as the target are characteristic of inflammatory diseases or of tumor tissue located underneath.

Suitable endothelial markers for inflammatory diseases are for example VCAMGPI, Class 1 MHC antigen, ICAM-1, VCAM-1, ELAM-1, E-selectin, P-selectin or VLA-4.

The cell surface molecules 4Ff2, EndoGlyx-1, endoglin (CD 105), the galectins and in particular endosialin are suitable as markers for tumor tissue located beneath the vascular endothelium and for angiogenesis caused by tumor growth and the associated changes to the vascular endothelium.

Lectins, fragments or derivatives of them, which specifically bind to the characteristic carbohydrate structures of the glycocalyx of endothelial cells modified by pathophysiological processes, act as the target-seeking unit for the conjugate according to the invention.

A monovalent lectin is preferably used for the conjugate, so that agglutination of the blood cells, e.g. of the erythrocytes, does not occur during the application.

Also the normal bodily functions should not be influenced or disturbed by the lectin used.

The lectins, fragments or derivatives of them used in the conjugate are of vegetable, animal, bacterial, viral or human origin.

Specific Examples of Suitable Lectins for the Invention are:

Lectins of Viruses:
Lectin of the rotavirus
Lectin of the vaccinia virus

Bacterial Lectins:
Adhesins, in particular the adhesin of Bordetella Pertussis
Family 13 of the carbohydrate-binding molecules
Comitin lectin from the bacteria lawn dictyostelium
Neuroaminidase of Vibrio Cholera

Lectins of Single Cells:
Lectin of entamoeba

Lectins from Plants:
LEA, DSA, GSA-1B₄, PHA, SBA, UEA-1, CSA-1, WGA, potato lectin (STA), barley lectin, LAA-1, MAA, GNA, DBA, jacalin, SCAman, ConA, ECA, PSA, RCA, LOLI, modeccin, abrin, viscun, ricin, ML-1, allium lectin

Animal Lectins:
BSCLT from Botryllus Schlosseri
BgSEL from Miomphalaria Glabrata
Ficolin/opsonin P35 lectin from the hedgehog
Echicetin from the snake
BjcuL from the snake
RVV-X from the snake
Galectin from the nematode Caenorhabditis Elegans
BmLBP from the silk worm
CCF-1 of invertebrates
Ly-49 of the mouse
Limulin
Limulus factor C
LFA
HPA

Human Lectins:

1) C-Type Lectins
Family of collectins: Clq, MBL, HSP-A, HSP-D, collectin 43, α-ficolin, β-ficolin, conglutinin, P35
Family of siglecs: siglec 1 (sialoadhesin), siglec 2 (CD22), siglec 3, siglec 4, siglec 5, siglec 6, siglec 7, siglec 8, siglec 9, siglec 10
Family of lecticans: aggrecan, versican, neurocan, brevican
Cytokines: IL-3, TFN-α, thrombomodulin, CD11b/CD18, CD66b, elastin/laminin binding protein, CD44, EN4, CD36, hevein, pseudohevein
Adhesion molecules: Galactosyl receptor, PECAM-1 (CD31), EpCAM, ICAM-1 (CD54), ELAM-1 (CD62E), VCAM-1 (CD106), CD72, vitronectin, LEC-CAM, in particular LEC-CAM-1
C-type lectins, which are associated with the immune system: NK cell domains such as Ly-49, CD23, or CD69, monocytic and macrophage lectin, CD72, T&B lymphoblastoid mucin-type lectin, P47, LSLCL gene product
Other lectins: Hepatic asialoglycoprotein receptor, tetranectin, P58/ERGIC-53, LOX-1, coagulation factors of IX/X binding protein, polycystin-1-C-type lectin, mucin-type proteins with C type lectin domains, myelin-associated glycoprotein, endosialin (TEM1), endoglyx, lipopolysaccharide-binding protein.

2) S-Type Lectins
S-Lac lectins
Galectins: galectin-1, galectin-3, galectin-9

Particularly preferred are the vegetable lectins, here particularly LEA, GSA-1B₄, UEA-1, ConA and WGA, the animal lectin LFA and the human bodily lectins, here particularly LOX-1, thrombomodulin, endosialin, endoglyx and galectin-1.

A further functional component of the conjugates according to the invention is a signal unit coupled to the lectin by suitable methods and which remains stable on the lectin under physiological conditions. This signal unit comprises a lanthanide ion, preferably a gadolinium ion or europium ion. The lanthanide ion is here preferably bound to a suitable chelator. Some unrestricted examples of suitable chelate-forming molecules or chelators are EDTA, DTPA (diethylenetriamine penta-acetic acid), DOTA (1,4,7,10-tetraazacyclododecane-N,N,N,N tetra-acetic acid), DFO (deferoxamine). DTPA or DFO is particularly preferred.

Chelator units, oligomerised or polymerised by suitable methods, can also be used as signal unit to achieve a higher metal ion burden in the conjugate. For example, suitable diols, such as ethylene glycol, 1,3-propylene glycol or N,N-bis-(2-hydroxyethylglycin), or diamines, such as ethylene diamine, 1,3-propylene diamine or 1,6 Hexamethyle diamine can be used as bifunctional, bridging reagents so that the chelator monomers are coupled to one another by ester or amide functions.

The number of the monomers coupled by the bifunctional units described above is n=2 to 20, preferably n=2 to 15, more preferably n=2 to 12 and very preferably n=3 to 10. Also naturally occurring polymers and fragments of them, such as for example chitosan, dextran or polylysine, which are linked in variable stochiometric ratios with the chelator units, can be used to increase the metal ion burden.

The monomer or oligomer chelator units of the signal unit can be covalently coupled to the lectin, fragment or derivative thereof and act at the same time as covalent linkers.

The coupling of the signal unit to the lectin, fragment or derivative thereof occurs through the use of a suitable functional group of the lectin without impairing its biological function. If required, this functional group can be introduced through modification of the native lectin. Also the use of a biotinylated lectin can be used for coupling an avidinylated signal unit. Preferably the coupling of the signal unit occurs via a free nitrogen function of the lysine in the lectin. In principle all known linker molecules, which facilitate a reliable transport of the signal unit to the target location and reliable dwelling of the signal unit at the target location, can be used as linkers for coupling a signal unit to the lectin. A suitable linker for coupling to the signal unit is non-toxic and neither impairs the biological behaviour and the specificity of the lectin, or fragment or derivative thereof, nor the activity of the signal unit to a significant degree. Suitable linkers can be selected in dependence of the type of substance intended for coupling and of the type of reactive functional group on the lectin. A range of linkers for the coupling of diagnostic and therapeutic substances to various functional groups on proteins are already known in the state of the art and can, where required, be adapted and used for the special conjugates according to the invention.

For the synthesis of the lectin linker conjugate the specific binding point of the lectin is protected where necessary by a suitable, temporary binding ligand which is to be removed again after successful coupling. In this respect, for example, specific binding oligo- or polysaccharides can be involved which can be removed again after successful coupling, for example, through affinity chromatography. This protective group may, for example, be chitobiose.

In a special aspect of the invention specifically exprimed lectins are detected on the surface of endothelial cells of the vascular vessel wall. Moreover, according to the invention divalent sugars are used, which on one hand possess a selective binding affinity with respect to the lectins on the endothelial cells and on the other hand possess a second affinity with respect to the lectin, or fragment or derivative thereof, of the target-seeking unit of the lectin conjugates according to the invention. Here, the divalent sugar can either be bound before the application to the specific binding point of the conjugated lectin or applied separately, so that the sugar first binds on the specifically exprimed lectin on the endothelial cell and is only then detected and bound by the conjugated lectin.

To achieve a therapeutic effect, the target-seeking unit, i.e. the lectin, or fragment or derivative thereof, or the conjugate from the target-seeking unit and the signal unit are provided with a therapeutic active substance in such a manner that the active substance can be released again without problem at the target location characterized by the target.

The therapeutic active substance is preferably a cytotoxic substance. This can, for example, be a metal complex such as cis-platinum or a suitable derivative thereof. Also other cytostatically effective substances, such as alkylating agents, antibiotics, antimetabolites, hormones or mitosis inhibitors can be used as active substances. Also growth factors, toxins, recombinant proteins or vectors for gene transfections can be used. With the use of radionuclides the radioactive particle is coupled to the lectin by a suitable chelator, such as for example DTPA or DFO. Also here, higher metal ion concentrations per mol of lectin can be achieved through the use of higher oligomerised or polymerised chelator units. In the case of non-metallic radionuclides, the radioactive element can be incorporated into suitable groups and thus coupled to the protein.

As radionuclides ¹²³I, ¹²⁵I, ¹³⁰I, ¹³¹I, ¹³³I, ¹³⁵I, ⁴⁷SC, ⁷²As ⁷²Se, ⁹⁰Y, ⁸⁸Y, ⁹⁷Ru, ¹⁰⁰Pd, ^{101m}Rh, ¹¹⁹Sb, ¹²⁸Ba, ¹⁹⁷Hg, ²¹¹At, ²¹²Bi, ²¹²Pd, ¹⁰⁹Pd ¹¹¹In, ⁶⁷Ga, ⁶⁸Ga, ⁶⁷Cu, ⁷⁵Br, ⁷⁶Br, ⁷⁷Br, ^{99m}Tc, ¹¹C, ¹³N, ¹⁵O and ¹⁸F can be used. Particularly preferred are ¹²⁵I, ¹¹¹In, ^{99m}Tc and ¹⁸F.

Preferably, the active substance is bound to the lectin, or the fragment or derivative thereof. The coupling of the active substance occurs via covalent linkers using a suitable functional group of the lectin without impairing its biological function. If required, this functional group can be introduced by modification of the native lectin, e.g. through chemical or genetic engineering methods. Preferably the coupling of the active substance occurs via a free nitrogen function of the lysine in the lectin.

In principle all known linker molecules, which facilitate a reliable transport of the active substance to the target location and a release of the active substance at the target location, can be used as linkers for coupling an active substance to the lectin. A suitable linker for coupling the signal unit or the active substance is non-toxic and neither impairs the biological behaviour and the specificity of the lectin, or fragment or derivative thereof, nor the activity of the active substance to a significant degree. Preferred linkers for the coupling of metal ions have already been mentioned above.

Other suitable linkers can be selected in dependence of the type of substance intended for coupling and of the type of reactive functional group on the lectin. A range of linkers for the coupling of diagnostic and therapeutic substances to various functional groups on proteins is already known in the state of the art and can, where required, be adapted and used for the special conjugates according to the invention.

If required here, the specific binding point of the lectin is protected by a suitable, temporarily binding ligand which is to be removed again after successful coupling. In this respect, for example, specifically binding oligo- or polysaccharides can be involved which can be removed again after successful coupling, for example, through affinity chromatography. This protective group may, for example, be chitobiose.

With the lectin active-substance conjugates according to the invention the lectin, fragment or derivative of the target-seeking unit is formed preferably such that the pathologically modified endothelial cell can first bind the lectin to its glycocalyx. The lectin can then be accepted into the cell and released again in the sub-endothelial region. A selective and targeted incorporation into the seat of the disease is then possible.

A therapeutic effect can also be achieved through a selective marking of the endothelial cell followed by transcytosis, so that a high active substance accumulation or concentration is obtained on the sub-endothelial side of the vascular vessel wall. The high accumulation then simplifies the passive transport of active substance in the direction of the diseased tissue by diffusion.

Preferably, pathophysiological conditions in the tissue to be treated are inflammatory or tumor diseases.

In a preferred embodiment at least one signal unit and at least one active substance are coupled to the same lectin. Due to the common coupling of a signal unit and an active substance to one and the same lectin, diagnostic tracking of the accumulation of active substance in the patient's body is possible.

In a special aspect of the invention the conjugates can only consist of a combination of the target-seeking unit, i.e. lectin, or a fragment or derivative thereof, and the therapeutic active substance. For this embodiment combinations of all the lectins mentioned above, or fragments and derivatives of them, with all the active substances mentioned above are possible. In this respect, particularly preferable are the lectins from the human body and in particular LOX-1, leucocyte and macrophage receptors, elastin/laminin-binding protein, CD11b/CD18, MBL, thrombomodulin, vitronectin and EpCAM.

The molar ratio between lectin, or a fragment or derivative thereof, and the respective signal unit coupled to the lectin or the respective coupled active substance can be varied for the optimum fulfilment of the special task of the respective lectin conjugate in an empirically determinable range. These types of optimisation experiments are in any case within the scope of the capabilities of the average person skilled in the art in this field.

The lectin, signal unit and optionally the active substance can be formed in separate synthesizing methods and combined as required. Due to this modular structure, a variation of the properties of the conjugates according to the invention, e.g. a change in the lectin specificity is easily possible.

In a special embodiment the lectin conjugates according to the invention can be immobilized on the surface of a polymer carrier or enclosed within a polymer carrier. Special examples of such polymer carriers are nano- or micro-particles based on polystyrene or chitosan, BSA/PLA (polylactic acid) micro-particles or latex particles, e.g. from polystyrene. The size of the polymer carrier here is selected such that the normal blood flow in the blood vessels is not disturbed by the presence of the lectin conjugate.

In this way high concentrations of the desired effector unit can be achieved at the target location.

The conjugates according to the invention and coupled to a signal unit can be used in various image-based diagnostic methods, preferably in MRI (“nuclear resonance imaging/Magnetic Resonance Imaging”) for the detection of specific targets, e.g. characteristic carbohydrate structures or specific exprimed lectins, on vascular endothelial cells or optionally on the tissue situated beneath. In a further aspect, the endothelium marking for MRI can also occur through a less selectively working lectin, so that the whole vascular endothelium is marked completely by the lectin signal unit conjugate, thus facilitating complete imaging of the blood vessel through the determination of the blood vessel volume in a given volume element.

### EXAMPLE 1

### Synthesis of LES-DTPA-Gd (Substance 1)

a) Production of Nitrilo-Acetic Acid Gadolinate, Tri-Sodium Salt (Gd(NTA)2Na3):
Gd₂O₃+6HCI→2GdCl₃+3H₂O
GdCl₃+2Na₃NTA→Gd(NTA)₂NA₃+3NaCl

2.10 g of Gd₂O₃ (5.8 mmol) are suspended in 0.046 g HCI (37%, 1.27 mmol) and heated to boiling point. For complete dissolving further HCI (37%) is added drop by drop until the boiling solution becomes clear. The GdCl₃ solution is diluted to 60 ml and then 6.38 g of Na₃NTA (23.2 mmol) are added at 40° C. The solution is made up to 100 ml and the pH value is adjusted to pH=6 by adding 3 M of NaOH.

b) Conjugation of DTPABA (Diethylene Triaminopenta-Acetic Acid-Bisanhydride)

10 mg of LEA (1.41×10⁻⁴ mmol) are dissolved in 0.22 ml of phosphate buffer (0.1 M, pH 7.4) in an Eppendorff tube. 11.2 mg of DTPABA (0.031 mmol, 220 times excess) are suspended in 0.028 ml of DMSO and then added in 4 portions to the protein solution. Between the individual additions, the pH value is adjusted to pH 8.5 by the addition of 3 M of NaOH. After the last addition the solution is allowed to stand for 1 hour at room temperature and shaken every 10 minutes.

c) Complexing of Gd(III)

To remove unbound DTPABA the reaction solution is separated using FPLC (Fast Protein Liquid Chromatography) through gel filtration with citrate buffer (0.1 M; pH 6.5). The protein fraction with the covalently bound DTPA is isolated. The 3 ml of protein solution obtained are supplemented with 0.030 ml of Gd(NTA)₂ solution (production as under a)) and stirred for 24 h at 4° C. Then the solution is lyophilized, dissolved in 0.5 ml of distilled water and again purified with FPLC to separate unbound Gd(NTA)₂ and free H₃NTA.

Product Properties:

MW_{lectin} 71.000 g/mol; the conjugate is characterized using AAS and protein determination.

### EXAMPLE 2

### LEA-DTPA-Gd, Encapsulated in Chitosan Nanoparticles

Sodium-bis(ethylhexyl)sulfosuccinate (0.03-0.1 M) is dissolved in 40 ml of n-hexane. To this solution 100 μl of 0.1% chitosan acetic acid solution, 200 μl LEA-DTPA-Gd solution (different concentrations), 10 μl of ammonia solution and 10 μl of 0.01-1.0% glutaraldehyde solution are added under constant stirring at room temperature. The reaction solution becomes homogeneous and clear. In this way chitosan nanoparticles form with encapsulated LEA-DTPA-Gd conjugate. In the next synthesizing step the solvent is removed on the rotary evaporator and the dry residue is resuspended in 5 ml of tris-Cl buffer (pH=7.4) in the ultrasonic bath. Then 1 ml of 30% CaCl₂ solution is added drop by drop. The activator precipitates in the form of calcium diethylhexyl-sulfosuccinate [Ca(DEHSS)₂]. The precipitate is centrifuged for 30 minutes at 4° C. and 5000 rpm. The pellet is discarded and the residue containing the nano-particles is isolated and centrifuged twice at 60000 rpm for 2 h each time. The isolated pellet (with the nanoparticles) is suspended in 5 ml of tris-Cl buffer (pH=7.4) and kept at 4° C.

The contrast medium thus produced is characterized by AAS, FACS, SEM.

### EXAMPLE 3

### Synthesis of LATEX-LEA-DTPA-Gd Under Different Conditions

I) Production of the Substances 3.1, 3.2, 3.3 Using Prepared LEA-DTPA-Gd Conjugate:

2 mg of polystyrene COOH (400 nm), 2 mg EDC [=1-(3-dimethylaminopropyl)-3-ethylcarbodiimide-hydrochloride] and 0.73 mg LEA-DTPA-Gd conjugate (refer to Example 1) (LEA-DTPA-Gd:EDC=1:1000) are dissolved in 0.4 ml of tridistilled water (or in phosphate buffer for Substance 3.3; LEA-DTPA-Gd:EDC=1:100). The reaction mixture is incubated for 18 h at room temperature. The solution is then centrifuged off and the residue containing the polystyrene LEA-DTPA-Gd conjugate is purified using FPLC. In the case of the Substances 3.1 and 3.3 no centrifuging takes place before the FPLC purification.

II) Production of the Conjugate with Prior Reaction of the Polymer with LEA (Substance 3.4):

The activator and the ionic impurities in the latex suspension are separated by centrifuging (3×10 min. at 4000 rpm in PBS). 36.0 mg of latex particles are resuspended in 4 ml of PBS. After the addition of 35.0 mg of EDC [1-(3-dimethylaminopropyl)-3-ethylcarbodiimide-hydrochloride] the reaction mixture is stirred for 3.5 h at room temperature. After removal of the activator by washing (centrifuged once for 3 min. at 4000 rpm), 10 mg of LEA are added and incubated overnight at room temperature. The reaction mixture is then centrifuged once at 4000 rpm to separate free lectin. Then the polymer is resuspended in 1 ml of PBS and kept at 4° C. The conjugation with DTPABA is carried out as the next working step. This occurs analogously to steps b) and c) in Example 1. The charging with gadolinium (III) according to Example 1c) occurs with 2×0.030 ml of Gd(NTA)2 solution.

### EXAMPLE 4

### Synthesis of PLA-LEA-Gadolinium Conjugates

Synthesis of BSA/PLA microparticles (diameter approx. 2 μm)

260 mg of poly-(D,L-lactic acid) are dissolved in 5 ml dichloromethane and emulsified in 80 ml of BSA solution (25% wt./vol.) at 24000 rpm for 3 min. using the Ultra Turrax T25. The resulting emulsion is stirred overnight at room temperature. Solid microparticles form which are collected by centrifuging (3300×g, 15 min.) and washed six times with tridistilled water. The suspensions are then kept at 4° C. The microparticle concentration is determined by the weighing of 1.5 ml of suspension after lyophilization.

Production of the Conjugates:

Synthesis of BSA/PLA-LEA-Gd (Substance 4.1)

Principle: The lectin is covalently bound to BSA/PLA microparticles. The microparticles are first activated with glutaraldehyde (25% aqueous solution) and then the incubation carried out with LEA as a second step.

50 mg of BSA/PLA microparticles are washed by centrifuging (3300×g, 10 min.) in PBS (10 mM, pH=7.4). The pellet is resuspended during vortexing in 1 ml of PBS. Then 1 ml of 25% aqueous glutaraldehyde solution is added and the mixture is shaken for 6 h to activate the amino groups. To separate the unreacted glutaraldehyde, the BSA/PLA suspension is centrifuged off and washed four times with PBS (10 mM, pH 7.4). Then 0.25 mg of LEA in PBS is added to 1 ml of BSA/PLA suspension. The reaction solution is incubated overnight at room temperature. The LEA microparticle conjugate is then centrifuged, suspended again in 1 ml of PBS and stored at 4° C.

The quantity of bound lectin is found with the difference method (lectin in the preparation/lectin in the supernatant after conjugation). The protein determination occurs according to the amido black method.

Conjugation with DTPA:

3.5 mg of DTPABA (0.009 mmol, 220 times excess) in 0.007 ml of DMSO are added to 0.5 ml of the microparticle suspension. The pH value is adjusted to 8.5 by adding 3 M NaOH. The suspension is allowed to stand for 1 h at room temperature and shaken every 10 mins.

Complexing of Gd(III):

The reaction mixture is centrifuged off for 3 minutes at 4000 rpm. The pellet is discarded and the supernatant containing the microparticles is separated with FPLC using citrate buffer (0.1 M; pH=6.5). The BSA/PLA-LEA fraction is isolated with the covalently bound DTPA. The 3 ml solution obtained is supplemented with 3×0.030 ml of Gd(NTA)₂ solution and stirred for 24 h at 4° C. Then the solution is lyophilized, dissolved in 0.5 ml of distilled water and again purified with FPLC to separate unbound Gd(NTA)₂ and free H₃NTA.

Synthesis of BSA-Gd/PLA-LEA-Gd (Substance 4.2):

First, the BSA-DTPABA-Gd conjugate required for this compound is produced as follows.

20 mg of BSA (0.3×10⁻³ mmol) are dissolved in 0.44 ml of HEPES buffer (0.1 M; pH=8.8) in an Eppendorff tube. 23.6 mg of DTPABA (0.066 mmol, 220 times excess) are suspended in 0.059 ml of DMSO and then added in three portions to the protein solution. Between the additions, the pH value is adjusted to pH=8.5 by the addition of 3 M of NaOH. After the last addition stirring is carried out for a further 2 h at room temperature.

To remove unbound DTPABA the reaction solution is separated using FPLC (Fast Protein Liquid Chromatography) through gel filtration with citrate buffer (0.1 M; pH=6.5). The protein fraction is isolated with the covalently bound DTPA. The 3 ml of protein solution obtained are supplemented with 0.056 ml of Gd(NTA)₂ solution (0.016 mmol) and stirred for 24 h at 4° C. Then the solution is lyophilized, dissolved in 0.5 ml of distilled water and again purified with FPLC to separate unbound Gd(NTA)₂ and free H₃NTA.

50 mg of BSA-DTPA-Gd/PLA microparticles are washed by centrifuging (3300×g, 10 min.) in PBS (10 mM, pH=7.4). The pellet is resuspended during vortexing in 1 ml of PBS. Then 1 ml of 25% aqueous glutaraldehyde solution is added and the mixture is shaken for 6 h to activate the amino groups. To separate the unreacted glutaraldehyde, the BSA-DTPA-Gd/PLA suspension is centrifuged off and washed four times with PBS (10 mM, pH 7.4). Then 0.25 mg of LEA are added to 1 ml of the BSA-DTPA-Gd/PLA suspension. The reaction solution is incubated overnight at room temperature. The LEA-BSA-DTPA-Gd microparticle conjugate is then centrifuged, suspended again in 0.5 ml of PBS and stored at 4° C.

Conjugation with DTPA:

3.5 mg of DTPABA (0.009 mmol, 220 times excess) in 0.007 ml of DMSO are added to the microparticle suspension (0.5 ml). The pH value is adjusted to 8.5 by adding 3 M NaOH. The suspension is allowed to stand for 1 h at room temperature and shaken every 10 mins.

Complexing of Gd(III):

The reaction mixture is centrifuged off for 3 minutes at 4000 rpm. The pellet is discarded and the supernatant containing the microparticles is separated with FPLC using citrate buffer (0.1 M; pH=6.5). The BSA/PLA-LEA fraction with the covalently bound DTPA is isolated. The 3 ml solution obtained is supplemented with 3×0.030 ml of Gd(NTA)₂ solution and stirred for 24 h at 4° C. Then the solution is lyophilizated, dissolved in 0.5 ml of distilled water and again purified with FPLC to separate unbound Gd(NTA)₂ and free H₃NTA.

The quantity of bound lectin is found with the difference method (lectin in the preparation/lectin in the supernatant after conjugation). The protein determination occurs according to the amido black method.

### EXAMPLE 5

### Immobilization of Lectin Conjugates on the Surface of a Polymer Carrier

As carriers, polycondensates of the chelatising compound DTPABA are produced with in each case [N,N-bis(2-hydroxyethyl)glycine)], ethylenediamine, hexamethylenediamine and ethylene glycol. The chain length of the polycondensates varies between n=5-9 units.

I. Polycondensation:

0.7146 g DTPABA (2 mmol) are dissolved in 10 ml of DMSO (abs.) in a 100 ml round flask with an opening for inert gas. 0.3264 g of bicine or bifunctional reagent (2 mmol) are added and the mixture stirred for 72 h under N₂. The polycondensate is precipitated in 10 times the amount of acetone, filtered and washed with acetone. The product is dissolved in water and again precipitated with acetone (10 times the amount), filtered and washed. Then it is dried at 40° C. under vacuum until a constant weight is obtained.

II. Conjugation of the Polycondensates with LEA (Lycopersicon Esculentum Agglutinin):

10 mg of LEA (1.41×10⁻⁴ mmol) are dissolved in 0.50 ml of phosphate buffer (0.1 M; pH 7.4) in an Eppendorff tube and then 10 mg of polycondensate are added. The pH value is adjusted to 8.5 by adding 3 M NaOH. Then the solution is allowed to stand for 1 h at room temperature and shaken every 10 mins.

III. Complexing of the Polycondensates with Gadolinium:

To remove unbound substances the reaction solutions are separated using FPLC (Fast Protein Liquid Chromatography) through gel filtration with citrate buffer (0.1 M; pH=6.5). The protein fraction with the covalently bound polycondensate is isolated. The 3 ml of protein solution obtained are supplemented with in each case 120 μl of Gd(NTA)₂ solution (0.016 mmol) and stirred for 24 h at 4° C. Then the solutions are lyophilized, dissolved in distilled water and again purified with FPLC to separate unbound Gd(NTA)₂ and free H₃NTA.

All compounds are characterized using ¹H-, ¹³C-NMR, mass spectrometry, AAS or protein determinations.

### EXAMPLE 6

### Use in MRI Diagnostics

a) Measurements of Relaxation

The dilution series of the various substances were produced in phosphate-buffered sodium chloride solution (PBS, pH=7.4); the concentrations were between 10⁻⁶ M and 1.0 M. PBS was used as the standard. The dissolved test substance was filled into an Eppendorff plastic tube as a 30 to 100 nM aliquot and positioned in the detector coil for the MRI measurements. The molar concentration was calculated in the case of the macromolecules on the basis of the carrier for which the analytical data are known and under consideration of the molar ratio between the carrier and the gadolinium. For the small molecules, such as Gd-DTPA or smaller polymers, the molar concentration was determined on the basis of the absolute molecular weight of the corresponding substance. In the case of the nanoparticles the concentration of the nanoparticle suspension and the absolute quantity of gadolinium, referred to the complete surface of the nanoparticle in the total volume, were used as the basis for the calculation of the concentration in the solution.

Relaxations were calculated in that the signal gain, which was measured in PBS, was compared with the values which were obtained in the gradually diluted concentration series of the Gd-DTPA compounds.

Results:

Substance 1 (LEA-DTPA-Gd): MRI data: SE (spin echo)=950% at 1×10⁻³ M, no SE at 1×10⁻⁵M.

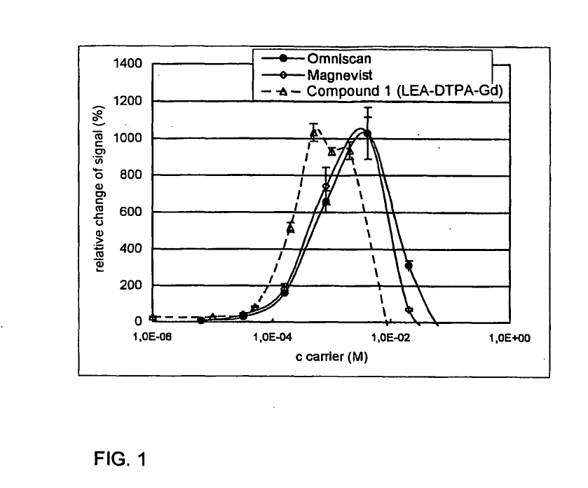

From the concentration/effect curve (FIG. 1) it can be seen that Compound 1 according to the invention causes an increase of signal for lower concentrations than omniscan and magnevist.

MRI images: With 1×10−4 M a weak image of the hepatic veins in the mouse (spin echo and flash 3D techniques).

Substance 2: MRI data: SE (spin echo)=110% at 1×10⁻⁴ M, no SE at 1×10⁻⁵ M. MRI images of the hepatic vein of the mouse using the spin echo technique.

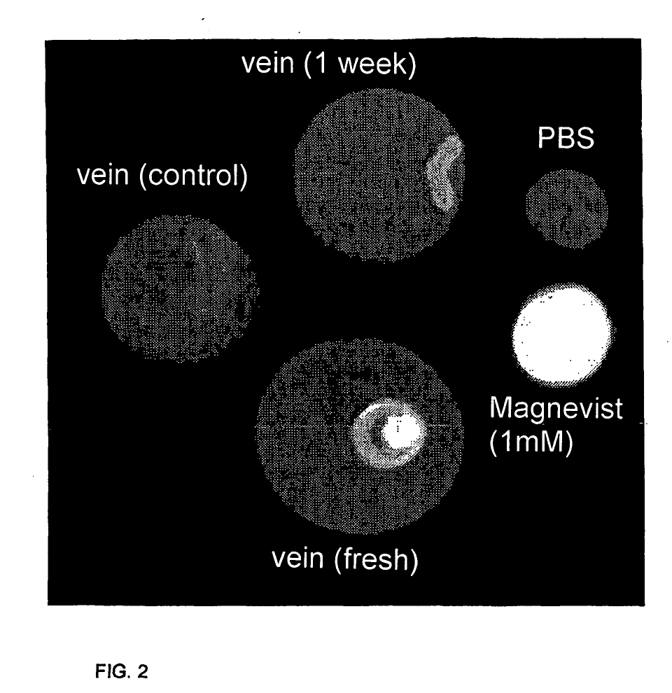

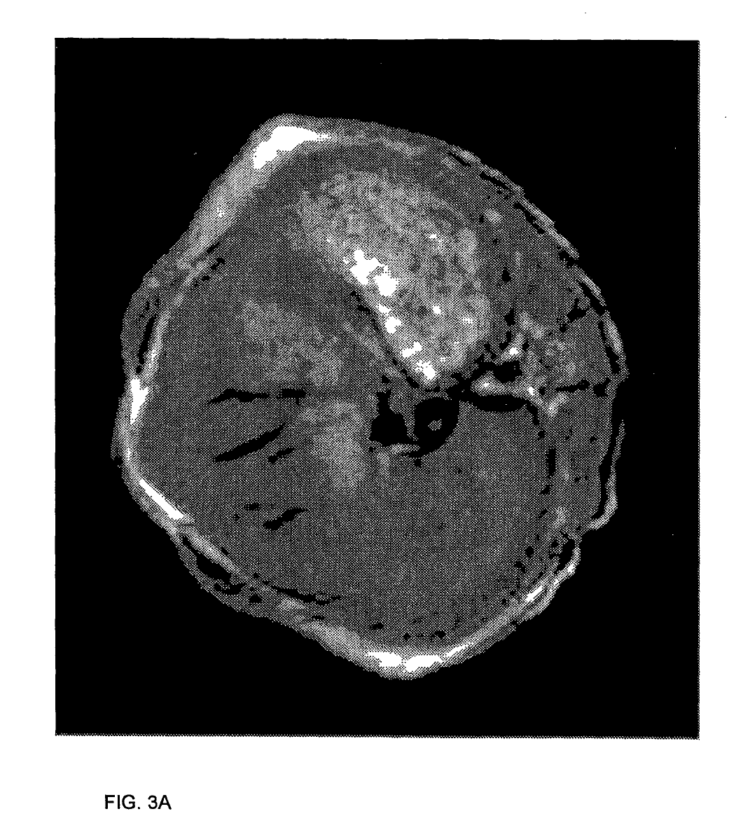

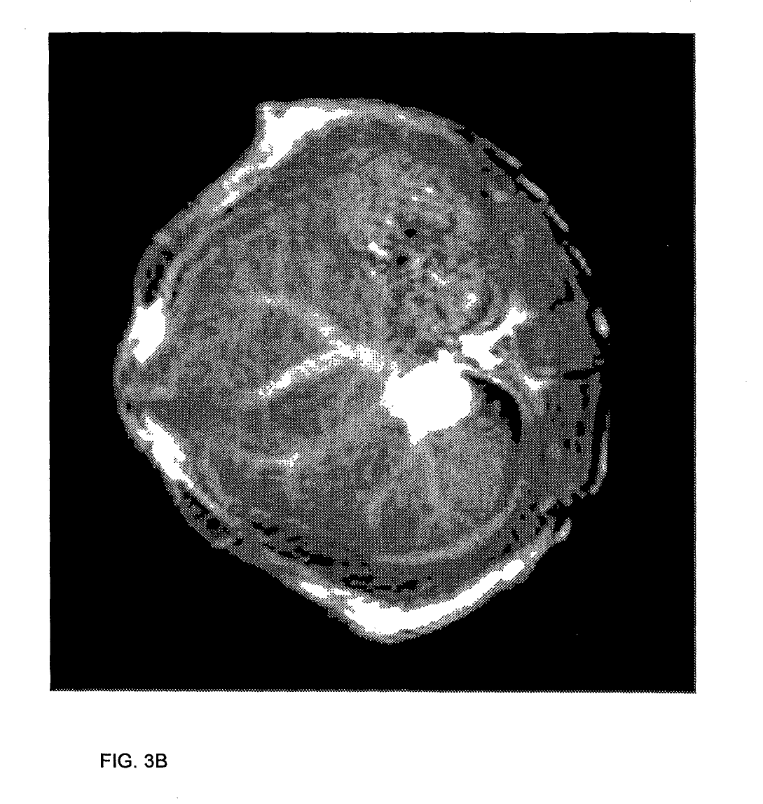

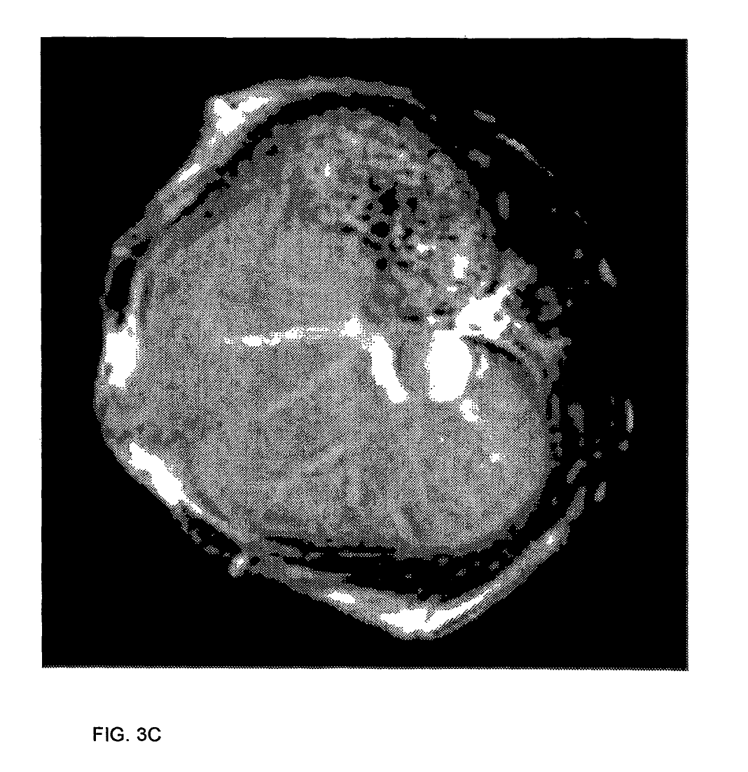

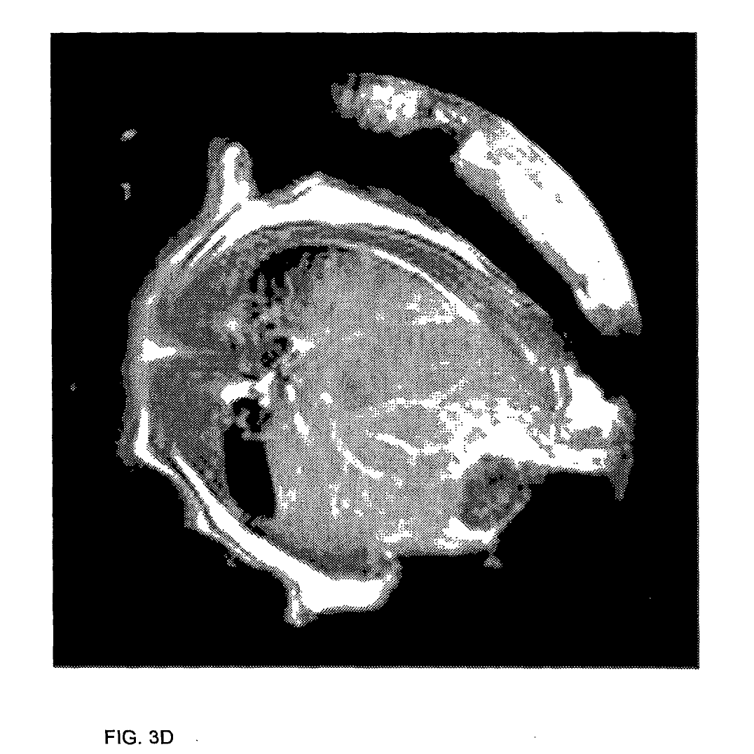

Substance 3.4: MRI images, refer to FIGS. 2 and 3.

Vein Representations

The human veins were removed during operative treatment of varicose veins. They were immediately placed in 2.5% glutaraldehyde, buffered with PBS. After the fixing in this solution for 18 to 24 hours at 4° C., the veins were transferred into the PBS and stored for up to 6 weeks at +4° C. During the operation the vein is turned inside out, which means that in contrast to the natural orientation under physiological conditions, the endothelium is situated on the outside.

Shortly before the test in the MRI, the veins were cut into 2 cm long fragments, placed in 0.15 M ammonium chloride solution for two hours up to one week at 4° C., then stored for at least one hour in PBS with 0.1% HSA, 0.1 mM of calcium ions and 0.1 mM of magnesium ions (PBS Inc.). Then the veins were placed for one hour in PBS Inc., which contained 10 mg/ml of latex-400-LEA-Gd (Substance 3.4). For a control, one vein was used which had been placed in PBS Inc. without the addition of additives.

MRI examinations were carried out while the vein segments were placed in PBS Inc. or in PBS Inc. with Substance 3.4. Then the veins were placed in 5 ml of PBS Inc. for rinsing, shaken for two minutes, then the vein was rinsed again with PBS Inc. and examined again in the MRI.

MRI experiments were carried out using the spin echo frequency and then using the FLASH-3D sequence.

The data were recorded in the form of digital images, such that quantitative data could be extracted from the images.

In FIG. 2 the results of the MRI measurement using the latex-LEA conjugate 3.4 in the human Vena saphena magna can be seen. These clearly show the deposition of the conjugate according to the invention on the endothelium of the Vena saphena magna.

Perfusion of the Mouse

Each mouse was narcotised with ketamine and Rompun, then small venflons were introduced into one of the carotid arteries and into one of the jugular veins. The mouse was then introduced into a falcon tube, which was open at one end, to ensure the entry of air and the falcon tube was positioned within the detector coil of the MRI unit. MRI measurements were carried out on various organs of the mouse (brain, intestine, liver, kidneys and lungs) before the perfusion with the test substance. The perfusion of 0.2 ml of the test substance within 60 seconds was tracked directly using MRI. After the MRI measurement the mouse was rinsed by perfusion with L15 in various volumes between 1.0 and 2.5 ml in the jugular vein. The mouse was immediately placed in the detector again and the MRI measurements were carried out again.

As an example of the use of the Latex conjugate 3.4, four MRI images of the mouse's liver are illustrated in FIGS. 3A to 3D. From these images the contrast between the blood vessels in the liver due to the use of a compound according to the invention can be clearly seen. Also, despite the complete rinsing of the blood vessel system of the mouse, the contrast medium remains in the blood vessels (FIGS. 3C and 3D).

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A conjugate, comprising at least one target-seeking unit, which specifically binds to receptors on a surface of endothelial cells, and at least one effector unit which is coupled to the unit by a linker and which comprises at least one signal unit and optionally at least one therapeutic active substance, wherein the target-seeking unit comprises a lectin or a fragment or derivative thereof, and wherein the lectin is not L-selectin and the signal unit comprises a lanthanide ion.

2. The conjugate according to claim 1, wherein the target seeking unit binds to characteristic carbohydrate structures of the glycocalyx of endothelial cells modified by pathophysiological processes.

3. The conjugate according to claim 1, wherein the target-seeking unit binds to specific lectins on the surface of endothelial cells.

4. The conjugate according to claim 1, wherein the target-seeking unit is a lectin of a viral, bacterial, vegetable, animal or human origin.

5. The conjugate according to claim 3, wherein a divalent sugar, which possesses a selective binding affinity for specific lectins on the surface of endothelial cells, is bound to the lectin, fragment or derivative thereof.

6. The conjugate according to claim 2, wherein the target-seeking unit binds to endothelial markers for inflammatory diseases.

7. The conjugate according to claim 6, wherein the target-seeking unit binds to VCAMPGPI, Class I MHC antigen, ICAM-1, VCAM-1, ELAM-1, E-selectin, P-selectin or VLA-4.

8. The conjugate according to claim 2, wherein the target-seeking unit binds to endothelial markers characteristic for tumor cells.

9. The conjugate according to claim 8, wherein the target-seeking unit binds to cell surface molecules 4Ff2, EndoGlyx-1, endoglin or endosialin.

10. The conjugate according to claim 1, wherein the target-seeking unit is monovalent.

11. The conjugate according to claim 1, wherein the lectin is LEA, a fucose-specific lectin such as UEA-1, a mannose-binding lectin such as MBL or ConA, a galactoside-binding lectin such as GSA-1B₄, a sialic acid-binding lectin such as LFA, or that the lectin is a lectin of the human body such as LOX-1, thrombomodulin, endosialin, endoglyx or galectin-1.

12. The conjugate according to claim 1, wherein the lanthanide ion is a gadolinium or europium ion.

13. The conjugate according to claim 1, wherein the effector unit comprises a therapeutic active substance which is selected from cytotoxic and cytostatic substances.

14. The conjugate according to claim 13, wherein the therapeutic active substance is selected from radionuclides, metal complexes, alkylating agents, antibiotics, antimetabolites, hormones, growth factors, mitosis inhibitors, toxins, recombinant proteins or vectors for gene transfections.

15. The conjugate according to claim 1, wherein the effector unit comprises a chelate-forming molecule.

16. The conjugate according to claim 15, wherein the chelate-forming molecule is ethylenediamine tetra-acetic acid (EDTA), diethylenetriamine penta-acetic acid (DTPA), 1,4,7,10-tetraazacyclododecane-N,N,N,N tetra-acetic acid (DOTA) or deferoxamine (DFO).

17. The conjugate according to claim 16, wherein the effector unit comprises several chelate-forming molecules, which are coupled together by a bifunctional bridging molecule.

18. The conjugate according to claim 17, wherein the bifunctional bridging molecule is selected from diols, e.g. ethylene glycol, 1,3-propylene glycol or N,N-bis-(2-hydroxyethylglycin), or diamines, e.g. ethylenediamine, 1,3-propylenediamine or 1,6-hexamethylenediamine.

19. The conjugate according to claim 16, wherein the chelator unit is coupled to naturally occurring polymers or fragments thereof, such as chitosan, dextran or polylysine.

20. The conjugate according to claim 1, wherein the conjugate is immobilized on the surface of a polymer carrier or is enclosed within a polymer carrier.

21. The conjugate according to claim 20, wherein the polymer carrier involves a nano- or microparticle based on polystyrene or chitosan, or it involves a BSA/PLA microparticle or latex particle.

22. The conjugate according to claim 1, wherein the conjugate is a LEA-DTPABA-Gd conjugate or LEA-DTPA-Gd conjugate.

23. A composition containing a conjugate according to claim 1, and optionally in addition therapeutic active substances, signal-generating components, physiologically acceptable carriers and auxiliary substances.

24. Use of the conjugate according to claim 1 in an image-generating method.

25. The use according to claim 24, wherein the image-generating method is MRI.

26. The use of the conjugate according to claim 1 in a therapeutic method.

27. The use of the conjugate according to claim 1 in a therapeutic method in which accumulation of an active substance in a patient's body is simultaneously traced.

28. A Manufacturing process of a conjugate according to claim 1, wherein
a) producing an aqueous solution of a complex salt of a lanthanide element,
b) conjugating the required lectin, fragment or derivative thereof with a chelate-forming molecule and removing unbound chelate-forming molecule,
c) reacting the conjugated lectin with the lanthanide salt solution to bind lanthanide ion to the conjugate via the chelate-forming molecule, and
d) optionally binding a therapeutic active substance to the lectin or to the conjugate according to step c).

29. The manufacture process according to claim 28, wherein the complex salt of the lanthanide element is nitrilo-acetic acid gadolinate tri-sodium salt (Gd(NTA)₂Na₃), the lectin is LEA and the chelate-forming molecule is selected from the group consisting of EDTA, DTPA, DTPABA and DFO.

30. A conjugate comprising at least one target-seeking unit, which bonds specifically to receptors on a surface of endothelial cells, and at least one effector unit coupled by a linker to the unit, which comprises at least one therapeutic active substance, wherein the target-seeking unit comprises a lectin or a fragment or derivative thereof, and wherein the lectin is not peanut lectin, lectin extract of orange peel, Maclura pomifera lectin, Dolichos biflorus agglutinin or soya bean agglutinin.
